(19) United States
(12) Patent Application Publication
(10) Pub. No.: US 2024/0341302 A1
(43) Pub. Date: Oct. 17, 2024

(54) METHOD TO PRESERVE NUCLEIC ACIDS IN WATER SAMPLES

(71) Applicant: UNIVERSITY OF CENTRAL FLORIDA RESEARCH FOUNDATION, INC., Orlando, FL (US)

(72) Inventor: Sean MOORE, Orlando, FL (US)

(21) Appl. No.: 18/682,519

(22) PCT Filed: Aug. 9, 2022
(86) PCT No.: PCT/US22/74690

§ 371 Date: Feb. 9, 2024

Related U.S. Application Data
(60) Provisional application No. 63/230,974, filed on Aug. 9, 2021.

(51) Int. Cl. A01N 1/02

(52) U.S. Cl. CPC A01N 1/0226

(57) ABSTRACT

Numerous fields of basic research, medicine, and technology require an understanding of the biological composition and diversity in water samples. A common approach to acquiring such information is to collect samples ‘in the field’ and to subsequently extract nucleic acids in a laboratory for sequence analyses. A challenge to this pipeline is the preservation of the abundance and quality of the nucleic acids present in and around the organisms because, once a sample is harvested, it is no longer connected to the environment that established it. Many organisms, particularly microbes, rapidly adapt to such environmental changes and the relative abundance and quality of nucleic acids within them can change in a manner of minutes. A common methodology intended to address this problem is to filter the water to collect microorganisms on site and then to store the filters in a frozen state until they are processed. This technique creates several problems: the filter systems are expensive, require a vacuum, and are cumbersome to operate; the microbes change their metabolisms as they are filtered; freezing the samples requires substantial infrastructure at the collection site and during transport; and freezing and thawing many organisms causes their cells to break open, which releases their nucleic acids. The disclosed invention circumvents these problems by allowing microorganisms and their nucleic acids to be rapidly preserved on site using simple collection vessels, without the need for refrigeration or freezing. The methodology allows for the subsequent recovery and processing of nucleic acids for downstream molecular analyses. Although the current embodiment was developed for sea water collection, it can be also be applied to other aqueous samples

### BACKGROUND

Some nucleic acid preservation composition and methods involve the collection of material from water samples using filtration systems and subsequent freezing. Such preservation methods require expensive and cumbersome infrastructures and disrupt the quality of the collected materials. Other preservation buffers involve the use of pre-dissolved ammonium salts for the preservation of solid biological materials or small amounts of aqueous samples. These preservation buffers do not address the inactivation of divalent cations. In addition, these preservation buffers require the transport and storage of large volumes of liquids to the collection sites and limit their application for sampling inhospitable environments, such as deep water. The preservation composition and method presented in this disclosure improves upon these reagents by avoiding these limitations.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows DNA extracted from preservation buffers, water, and the original sample.

FIG. 2 is a graph showing the UV absorbance spectra of purified nucleic acid.

FIG. 3 is a graph showing the DNA concentrations in extracted samples.

FIG. 4 is a agarose gel showing PCR tests of two extracts from seawater samples.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference. Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about” generally refers to a range of numbers that one of skill in the art would consider equivalent to the recited value (i.e., having the same function or result). In a specific embodiment, the term “about” includes a stated numerical value as well as a value that is +/−15% of the stated numerical value. For example, about 5.75 M includes 5.75 molar as well as 6.61 M and 4.89 M, and all 1/10 values in between. In many instances, the term “about” may include numbers that are rounded to the nearest significant figure.

The term “ammonium sulfate” refers to an inorganic sulfate salt obtained by reaction of sulfuric acid with two equivalents of ammonia. A high-melting (decomposes above 280° C.) white solid which is very soluble in water (70.6 g/100 g water at 0° C.; 103.8 g/100 g water at 100° C.), it is widely used as a fertilizer for alkaline soils. It has a role as a fertilizer. It is an ammonium salt and an inorganic sulfate salt.

The terms “biological material” or “biological sample”, refers to any organisms (including human, animal, or plant pathogens), human products (blood, tissues, bodily fluids, clinical specimens), animals (animal carcasses, or animal products including tissues, cells, blood, or other bodily fluids), recombinant or synthetic DNA/RNA (plasmids, cloned materials, oligonucleotides, siRNA), viral vectors (e.g., lentivirus, retrovirus, adenovirus, AAV), or genetically-modified organisms (animals, microorganisms, plants, insects, cells/cell lines). Samples can be taken from a water source such as an environmental source (river, pond, ocean, or lake), industrial source, or clinical source (human, animal, or laboratory).

The term “divalent cation” refers to a cation with electron valence of 2+. This type of ion may form two chemical bonds with anions. A magnesium ion, Mg²⁺ is a divalent cation. All of the alkaline earth metals (group 2) form divalent cations.

The terms “chelator” or “chelating agent” refers to chemical compounds whose structures permit the attachment of their two or more donor atoms (or sites) to the same metal ion simultaneously and produce one or more rings. These molecules are also called “chelates” or chelating groups, and the formation of rings is called “chelation.” These metal complexes have the ability to resolve into optically active (R&L) forms. Stability of metal complexes differs with pattern of complex formation and difference in stability becomes more relevant in the increasingly dilute solutions in biological systems such as serum or tissue. The process of chelation depends on both the nature and the properties of the metal and those of the chelating agent, such as ionic diameter, ring size and deformability, and hardness or softness of the electron donors and acceptors.

The term “collection tube” refers to any suitable containment method or device for containing a water sample of biological materials.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “preservation” or “preserve” refers to methods to prevent decomposition or deterioration of samples by enzymatic activity or by undesirable chemical changes. In general, preservation is implemented in two modes, chemical and physical. Chemical preservation entails adding chemical compounds to the product. Physical preservation entails processes such as refrigeration or drying. Chemical preservation and physical preservation techniques are sometimes combined.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “pH buffer” or “buffer” refers to a solution containing both a weak acid (HA) and its conjugate base (A⁻). Buffer solutions are used as a means of keeping pH at a nearly constant value in a wide variety of chemical applications. In nature, there are many systems that use buffering for pH regulation. In this embodiment, sodium citrate is used as a pH buffer in the composition.

The term “solid” refers to a composition that is firm and stable in shape; not liquid, fluid, or gas. In some embodiments solid refers to a dry powder material.

The terms “sterile” refers to a nearly contaminate-free and bacteria-free environment for preventing mixture of materials other than materials collected. In a preferred embodiment, the reagents or collection tubes do not contain nucleic acids to a degree that they would contaminate the samples. The term “sterilization” refers to any process that removes, kills, or deactivates all forms of life for establishing a sterile environment.

### DETAILED DESCRIPTION

It was found that certain salts can stabilize and precipitate biological materials, including proteins, nucleic acids, and cell bodies. In a preferred embodiment, a sufficient weight of ammonium sulfate (AmSO4), typically in solid form, is added to a collection vessel such that when an aqueous sample is added and the AmSO4 it dissolves into it, the salt nears its saturation limit and promotes the aggregation and preservation of biological materials contained within it. In addition, the AmSO4 portion is augmented by the addition of a low pH buffer (sodium citrate) to reduce damage to ribonucleic acids (RNA), and also a chelator of divalent cations (ethylenediaminetetraacetic acid, EDTA) to preserve deoxyribonucleic acids (DNA) and RNA, as RNA is particularly sensitive to certain metals, especially at higher temperatures. The EDTA concentration is of sufficient quantity to chelate the divalent cations present in the water sample. Once transferred to a laboratory, the material can be collected by centrifugation or filtration and then processed to recover nucleic acids. The collection vessels are stable, present a low hazard and do not contain liquids that can leak prior to collection. The use of a solid precipitant allows for a large volume of water sample to be collected. The EDTA concentration can be high enough to chelate divalent cations present in sea water, and the solid preservative can be present in a collection vessel as the initial sample is collected, even at great depths.

Several formulations have been constructed and evaluated for efficacy. The chemical formulation does not interfere with the subsequent extraction of DNA or with the extraction of total nucleic acids from intact bacterial cells. The process has been tested on sea water samples, and the recovered nucleic acids contain RNA and DNA. Further, the recovered DNA is of sufficient quality to serve as templates for polymerase chain reactions (PCR). Using PCR, it was demonstrated that the recovered DNA contained the chromosomes of several species of bacteria, which are the dominant microbial entity in sea water. It has been found that high quality ‘next-generation’ sequencing data can be obtained from such samples.

### Formulations

In some preferred embodiments, the preservation composition comprises a salt that precipitates nucleic acid in the sample along with the cellular protein. In some embodiments, the salt is a sulfate salt, for example, ammonium sulfate, ammonium bisulfate, cesium sulfate, cadmium sulfate, cesium iron (II) sulfate, chromium (III) sulfate, cobalt (II) sulfate, copper (II) sulfate, lithium sulfate, magnesium sulfate, manganese sulfate, potassium sulfate, sodium sulfate, or zinc sulfate. In a preferred embodiment, the salt is ammonium sulfate.

In preservation composition comprising salt, the salt is typically present in an amount sufficient to precipitate the nucleic acids in the sample along with the cellular protein. The salt is typically present in an amount between 5.75 M and the saturating concentration of the salt.

Specifically, salt concentrations of 4.54 M to 6.05 M may be used, and the amount may be a range defined between any two of these amounts.

The present invention is not limited to the use of ammonium sulfate, and other salts or compounds will also be useful in protecting nucleic acids and proteins in tissue samples and cell samples.

In certain embodiments the preservation composition may comprise a chelator of divalent cations, for example EDTA, sodium salicylate, methoxy salicylates, EGTA, HEDTA, or NTA. The chelator is typically present in an amount sufficient to assist in the removal of metals for the protection of the nucleic acids and proteins in the sample. In a preferred embodiment, the chelator is EDTA. The chelator is typically present in an amount of 37.5 mM. Specifically, amounts of about 10 mM to 55 mM to may be used.

Typically, the preservation composition comprises a buffer so that a constant pH can be maintained. For example, the buffer can be sodium citrate, sodium acetate, potassium citrate, potassium acetate, or citric acid. In a preferred embodiment, the buffer is sodium citrate. Typically, the buffer is typically present in an amount of 15-100 mM.

### Composition of Kit

The disclosure also contemplates kits for preserving nucleic acids and proteins within a sample. The solid components of the present disclosure can be prepared to yield the desired final component concentrations in solution when added to an aqueous sample. The solid components can further be provided as powders, tablets, pills or other suitable formulations that provide the desired properties of a preservation composition. Solid components can be directly added to a sample, added to a sample/liquid mixture, or present in a collection vessel prior to collection of a sample or sample/liquid mixture. The addition of excipients and bulking agents such as mannitol, lactose, starch, cellulose, and the like, to provide desired solid characteristics (i.e., improved solubility, storage stability, particle dispersion) are also considered in the formulation of powders, tablets and pills. The solid components of the present invention can be added prior to sample collection, after sample collection or any combination thereof.

Certain advantages of using a solid component preservation composition are weight savings in storage and transport, spills of solid components are less likely and reduced volume savings (i.e., preserving samples with dry reagents would minimize the final volume of the sample).

In one embodiment of the disclosure, pre-measured aliquots of a solid preservation composition can be loaded into sample collection vessels, and an appropriate volume of a sample containing intact cells, biofilms(s), tissue(s), nucleic acids or proteins added. The collection vessel would then be agitated, dissolving any solid components of the preservation composition, minimizing operator exposure to a sample. For example, a solid preservation composition could be any of the salts previously described. Thus, in particular embodiments of the disclosure, stabilizing nucleic acids or proteins in environmental water samples, clinical fluids, or industrial water systems for evaluation of the abundance and diversity of microorganisms is contemplated.

In one example, a tube for collecting specimen such as water sample containing microbes or pathogens is supplied with pre-measured aliquots of a preservation composition. Immediately following collection specimen, the tube is agitated, admixing the specimen containing nucleic acids or proteins (i.e., sample) with the preservation composition. The tube containing the specimen and the preservation composition are stored, until the specimen is brought to a suitable analysis site. The nucleic acids and proteins from the specimen are extracted from the preservation composition for further experimentation or analysis.

In one example, a vial for collecting specimen such as urine or blood could be supplied with pre-measured aliquots of a preservation composition. Immediately following collection of said specimen, the vial could be agitated, admixing the specimen containing nucleic acids or proteins (i.e., sample) with the preservation composition.

### Example

The preservation composition and method presented in this disclosure improves upon previous buffers and methods by preserves nucleic acids in water/solid samples in the field without need for refrigeration. The embodiments of the present disclosure preserves samples containing cells such as, but not limited to, microbial cells, blood cells, among many other types of cells. The new method to extract DNA from preserved samples was developed and validated by various methods. The method was evaluated by assessing the quality and quantity of extracted nucleic acids from preserved samples.

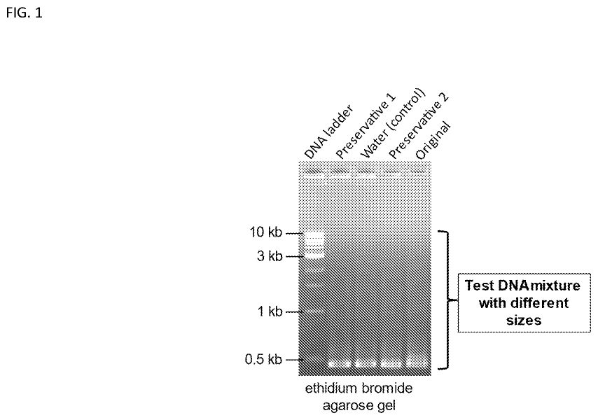

To start, it was evaluated for the ability to recover free DNA that was spiked into solutions prepared from the dry preservative. For this test, three 170 μL preservative samples were prepared by adding liquids to the dry preservative: ‘Preservative 1’ received a bacterial growth medium (“LB”: 10 g tryptone, 5 g yeast extract, 5 g NaCl (˜85 mM NaCl)); ‘Preservative 2’ contained the bacterial growth medium and an additional amount of NaCl to make the final concentration in a 200 μL mixture 500 mM (to mimic sea water); and another sample received pure water as a control. To these mixtures, 30 μL of a solution containing DNA with sizes ranging from −450 to 10,000 basepairs was added to make a final volume of 200 μL. Then the DNA was extracted and purified using silica columns and run on an agarose gel to compare the recoveries to an unprocessed aliquot of the DNA mixture (‘Original’) (FIG. 1). All recovered samples contained DNA with the same abundance and size distribution as the original sample.

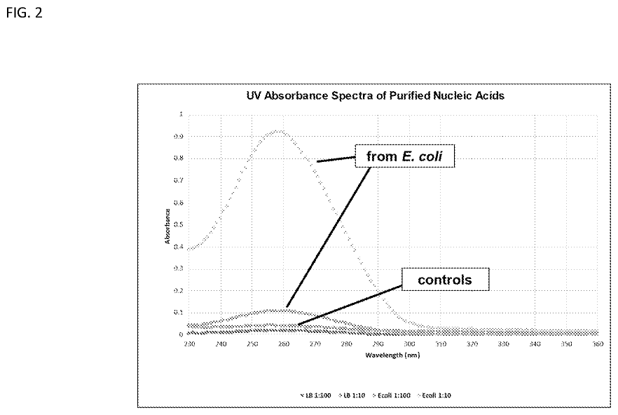

Next the ability to extract DNA from intact microbes suspended in the preservative was tested. Stationary phase cultures of E. coli grown in LB were diluted either 1:10 or 1:100 in fresh LB and then those liquids were added to solid preservative and stored overnight at room temperature. The bacteria were then recovered by centrifugation and total nucleic acids were extracted using zirconia bead ablation (a mechanical cell disruption) followed by silica column purification. The total recovered nucleic acids were then measured using UV absorbance (FIG. 2). The 1:10 E. coli dilution showed the greatest absorbance as expected, and the 1:100 E. coli dilution had an absorbance that was proportional to the dilution. The controls contained LB medium alone and they showed almost no absorbance.

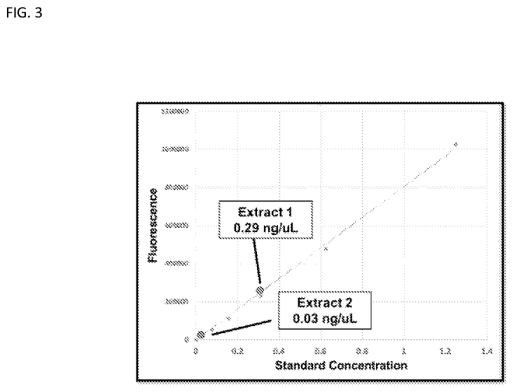

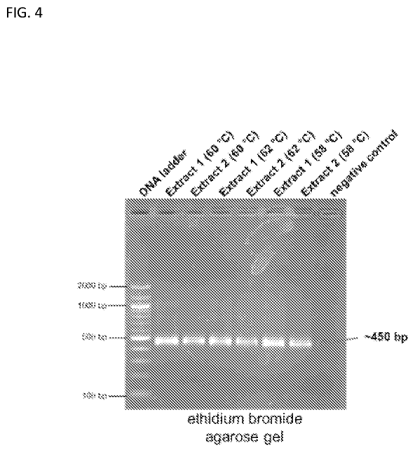

A preferred embodiment of the new preservative method is for extracting DNA from sea water samples for use in DNA amplification or sequencing projects. Because the yields from these samples are generally too low to be measured using UV absorbance, the concentration of DNA in the extracted total nucleic acids from two preserved sea water samples was measured using a DNA-specific fluorescent dye. A standard curve was established to calculate the concentration of the extracted DNA. About 0.29 ng/μL of DNA was extracted from the first sea water sample, and 0.03 ng/μL of DNA was extracted from the second (FIG. 3). To evaluate the quality of these extracts, they were used as templates in polymerase chain reactions (PCR). The PCR was run using primers that amplify the V3-V4 hypervariable regions of bacterial 16S rRNA genes, which are commonly used for establishing bacterial diversity and abundance. Different annealing temperatures were also evaluated, and the amplicons were run on an agarose gel. PCR amplicons formed at all three annealing temperatures and all amplicons were the correct size (FIG. 4) The DNA extraction was thorough, and ample DNA was extracted for this PCR method. The negative control showed a lack of bacterial genome contamination. Preliminary characterizations of these and other PCR amplicons obtained from sea water using this preservative method revealed they contain sequences from hundreds of established ocean-dwelling bacteria.

What is claimed is:

1. A biological material preservation composition comprised of ammonium sulfate (AmSO4), a pH buffer, and a chelator.

2. The composition of claim 1, wherein the composition comprises from about 4.54 M to 6.05 M of AmSO4.

3. The composition of claim 2, wherein the composition comprises about 5.75 M of AmSO4.

4. The composition of any of claims 1-3, wherein the pH buffer is selected from the group comprised of sodium citrate, sodium acetate, potassium citrate, potassium acetate, or citric acid.

5. The composition of claim 4, wherein the pH buffer is sodium citrate.

6. The composition of claim 5, wherein the composition comprises about 15-100 mM sodium citrate.

7. The composition of claim 6, wherein the composition comprises about 25 mM sodium citrate.

8. The composition of any of claims 1-7, wherein the chelator is selected from the group comprised of EDTA, sodium salicylate, methoxy salicylates, EGTA, HEDTA, or NTA.

9. The composition of claim 8, wherein the chelator is EDTA.

10. The composition of claim 9, wherein the composition comprises from about 10 mM to 55 mM of EDTA.

11. The composition of claim 10, wherein the composition comprises 37.5 mM EDTA.

12. The composition of any of claims 1-11, wherein the composition is a solid.

13. A method for preserving biological material in a water sample comprised i) colleting a water sample and ii) dissolving the composition of claims 1-12 into the water sample, whereby the method preserves biological material contained in the water sample.

14. The method of claim 13, wherein the biological material comprises nucleic acids, proteins, and other macromolecules.

15. The method of any of claims 13-14, wherein the biological material is preserved at about 20 to 22° C.

16. The method of any of claims 13-15, wherein any divalent cations are chelated from the water sample.

17. The method of any of claims 13-16, wherein the water sample is collected from an environmental, a clinical, or an industrial source.

18. A kit for preserving biological material comprised of the composition of claims 1-12 placed in a collection tube.

19. The kit of claim 18, wherein the collection tube is sterilized.
